(19) United States
(12) Patent Application Publication
(10) Pub. No.: US 2009/0123992 A1
(43) Pub. Date: May 14, 2009

(54) Shape-Shifting Vitrification Device

(76) Inventor: Milton Chin, Trumbull, CT (US)

Correspondence Address: Markets, Patents & Alliances LLC, 30 Glen Terrace, Stamford, CT, 06906-1401, US

(21) Appl. No.: 12/267,708

(22) Filed: Nov. 10, 2008

Related U.S. Application Data
(60) Provisional application No. 60/987,110, filed on Nov. 12, 2007.

(51) Int. Cl. C12N 1/04; C12M 1/00

(52) U.S. Cl. 435/260; 435/307.1

(57) ABSTRACT

This invention is a storage device (cryocontainer) for the vitrification method of cryopreservation that uses shape memory materials to create a novel shape-shifting feature in which the relevant heat transfer zone of the cryocontainer can be thermally morphed between a shape conducive to biological specimen handling and to a shape conducive to rapid heat transfer. This feature utilizes the temperature induced phase transformation of shape memory materials. The temperature inducement occurs naturally within the normal temperature changes that occur during vitrification.

### CROSS-REFERENCE TO RELATED APPLICATIONS

This application claims priority to U.S. provisional patent application entitled “Shape Memory Vitrification Cryocontainer”, Ser. No. 60/987,110 filed on Nov. 12, 2007. Said provisional application is incorporated herein by reference.

### TECHNICAL FIELD

This invention is in the field of devices for the cryopreservation of biological specimens.

### BACKGROUND

Cryopreservation is practiced in the life sciences for the purpose of halting biological activity in valuable cell(s) for an extended period of time. One factor in the success of cryopreservation is reducing or eliminating the deleterious effect of ice crystal formation. Sophisticated methods are needed to thwart the natural tendency of water to freeze into ice during cryopreservation.

### Cryopreservation

One method of minimizing ice crystal formation is called “slow-freeze.” The initial step in slow-freeze is to dehydrate a cell or cells with an aqueous solution (“slow-freeze media”) containing permeating and non-permeating cryoprotectants (“CPA”). The cell or cells, together with a small quantity of slow-freeze media, comprise the “biological specimen.” The biological specimen is then placed in a suitable cryocontainer, i.e. a container suitable for use at cryogenic temperatures. As used herein, “cryogenic temperatures” means temperatures colder than −80° C. Slow-freeze cryopreservation entails chilling the biological specimen from room temperature to its ultimate cryogenic storage temperature that is typically −196° C., the atmospheric boiling point of liquid nitrogen (“LN2”). For a portion of this temperature range, from approximately −6° C. down to −30° C., the chilling rate is precisely controlled to 0.1-0.3° C./minute by a programmable freezer. Chilling from −30° C. to −196° C. is achieved by plunging the cryocontainer in LN2. Slow-freeze processes take 2-3 hours to complete, hence the name. By this process, ice crystals do form in the CPA surrounding the cell or cells, and minimally within the cell or cells. Slow-freeze is effective in cells with low water content such as embryos and sperm, but does not perform as well in high water content cells such as oocytes and blastocysts. This deficiency, high equipment cost, and the high consumption of time have led to the development of an alternative cryopreservation method called vitrification.

### Vitrification

Vitrification differs from slow-freeze in that it seeks to avoid the formation of cell-damaging ice altogether. Similar to slow-freeze, the first step in vitrification is to dehydrate the cell or cells as much as possible using CPA containing fluids called “vitrification media.” The biological specimen (same definition as slow-freeze) is then rapidly chilled by immersion in a cryogenic fluid such as LN2. With a proper combination of chilling speed and CPA concentration, intracellular water will attain a solid, innocuous, glassy (vitreous) state rather than an orderly, damaging, crystalline ice state. Vitrification can be described as a rapid increase in fluid viscosity that traps the water molecules in a random orientation. Vitrification media, however, contain higher levels of CPA than slow-freeze media and are toxic to cells except in the vitreous state. Therefore, the time exposure of cells to vitrification media during dehydration and thawing (called “warming” since ice is not formed) must be carefully controlled to avoid cellular injury. The end point of vitrification and slow-freeze is the same: long term storage in a cryogen such as LN2.

If a chilling speed of 106° C./minute were possible, vitrification could be achieved with no cryoprotectants at all. Extremely toxic vitrification media, with 60% w/w CPA concentration, can be vitrified with ordinary chilling speed. Commercial vitrification media have CPA formulations and minimum enabling chilling speeds between these boundaries. The inverse relationship between CPA concentration and minimum enabling chilling speed is well known. The key to minimizing the toxic effects of vitrification media is to minimize its CPA concentration. Therefore, it is desirable to chill quickly; the faster the better. Given this, a natural initial discovery in this field was to directly plunge the biological specimen into LN2 to achieve rapid chilling. Carrier devices to enable direct plunge were created to facilitate and control this process.

Examples are: electron microscopy grids, open pulled straws, Cryoloop™, nylon mesh, and Cryotop. Cryoloop is a trademark of Hampton Research. These devices are classified as “open carriers” in that the biological specimen is in direct contact with the chilling cryogen, typically LN2. Open carriers also enabled rapid warming of the biological specimen.

LN2, however, is not aseptic. It may contain bacterial and fungal species, which are viable upon warming. Furthermore, it has been reported that vitrified cells held in long term storage in LN2 could be infected by viral pathogens artificially placed in said LN2. Hence, there is the potential for infection of biological specimens vitrified in open carriers.

The potential of infection has led to the development of closed cryocontainers where the biological specimen is placed in a cryocontainer and sealed before chilling in LN2. The cryocontainer also serves as a storage device to isolate it from pathogen-containing cryogen during long-term storage. But the very surfaces that protect the biological specimen also impede the removal of heat during vitrification and reduce chilling and warming speeds. Development of an effective closed cryocontainer for vitrification has proven to be a difficult challenge due to this conflict of purpose.

FIG. 1 illustrates the relationship, 100, between five competing design constraints of an effective closed cryocontainer. These constraints are “Safe Vitrification Media”, “Rapid Chilling and Warming Speeds”, “Aseptic Cell Environment”, “Physical Protection of Specimen” and “Ease of Use.” Vitrification requires rapid chilling and warming speed, the higher the better. The available chilling speed determines the CPA level in the vitrification media one can safely use without poisoning the cell. Arrow 102 indicates that these two factors are interrelated. The closed cryocontainer must maintain the biological specimen in an aseptic environment. It must remain this way during long term storage. The cryocontainer must be rugged enough to maintain its physical integrity during both normal and accidental rough handling (e.g. dropping). Closed cryocontainers must allow technicians of normal training to process biological specimens without undo frustration and must be able to tolerate minor errors in technique.

### Limitations of Current Cryocontainers for Vitrification

U.S. Pat. No. 7,316,896, “Egg freezing and storing tool and method”, ('896 Device) describes a closed cryocontainer for vitrification. This device comprises a fine plastic tube (nominally 0.25 mm OD and a wall thickness of 0.02 mm). A typical biological specimen will contain a human oocyte having an OD of 0.125 mm. It is dehydrated with vitrification media and then drawn into the tube. Then both ends of the tube are heat-sealed to create an aseptic container. Time is of the essence during loading since exposure to the toxic vitrification media at room temperature must be limited. Any delay in vitrifying the biological specimen may lead to cellular damage due to overexposure to warm CPA in the vitrification media. Because of the extremely small size of the '896 Device, however, the act of loading the biological specimen into it is not easy. The fineness of the '896 Device also raises questions as to its ruggedness in normal handling. Furthermore, since one of the heat seals is created very close to the biological specimen, there are concerns that the heat will injure the cell.

US Patent Application 2008/0220507, “Kit for Packaging Predetermined Volume of Substance to be Preserved by Cryogenic Vitrification”, ('507 Device) describes a tube-within-a-tube closed cryocontainer concept. Both tubes are fabricated from plastic. The inner tube is modified to create a channel at one end upon which the biological specimen is placed. The loaded inner tube is then placed within the outer tube. The outer tube is then heat-sealed at the loading end to create an aseptic cryocontainer. The '507 Device has dimensions that are an order of magnitude larger than the '896 Device and consequently is more user friendly. In order for the loaded inner tube to be placed within the outer tube, however, there must be some clearance between the two. Thus there is an air gap between the biological specimen and the outer tube. Air has low thermal conductivity and hence effectively insulates the biological specimen from the cryogen. The '507 Device exhibits relatively slow chilling rates which requires higher CPA levels.

International Patent Application WO 07/120829, “Methods of the cryopreservation of mammalian cells”, ('829 Device) describes the use of ultrafine tubes for vitrification. One embodiment of the '829 Device is an ultrafine microcapillary quartz tube. Biological specimens can be drawn into such a device and vitrified. Due to the exceedingly thin wall sections (10 microns) and high thermal conductivity of quartz, as compared to plastics, the inventors claim that the '829 Device will have a high (greater than 30,000° C./minute) chilling rate. The small size and thin walls however, imply a very fragile container and there is no indication as to how an aseptic seal is made.

Since 1984, vitrification has been used to cryopreserve human cells. However, its slow-freeze counterpart is still the dominant cryopreservation method. It is felt that the lack of a suitable cryocontainer has limited the practice of vitrification. Prior art cryocontainers are compromises that ignore one or more design constraints of FIG. 1 in favor of others.

### SUMMARY OF THE INVENTION

The Summary of the Invention is provided as a guide to understanding the invention. It does not necessarily describe the most generic embodiment of the invention or all species of the invention disclosed herein.

### Improved Cryocontainer for Vitrification

The purpose of this invention is to provide an improved closed vitrification cryocontainer. The device contemplated by this invention holistically incorporates all five attributes of FIG. 1. Rather than seeking solutions in increasingly smaller and fragile cryocontainers, this invention goes in a new direction and utilizes the features of shape memory materials to create a new design.

The present invention comprises a closed vitrification cryocontainer comprising deformable walls to achieve both ease of loading and unloading and rapid chilling. This feature utilizes the unique material characteristics of shape memory materials. Shape memory materials exist in two crystallographic structures: high temperature austenite and low temperature martensite. The austenite phase is characterized by stiffness and superelastic properties. The martensite phase is soft and malleable. The shape of an object in its austenite phase is referred to as the “memorized shape.” If a shape memory material is cooled from its austenite phase to its martensite phase and then deformed, it will return to its austenite shape when it is heated back into its austenite phase. Shuttling between these two phases enables design options that can advantageously be incorporated into the present invention.

### BRIEF DESCRIPTION OF DRAWINGS

FIG. 1 is a diagram that shows the design attributes required for a vitrification cryocontainer.

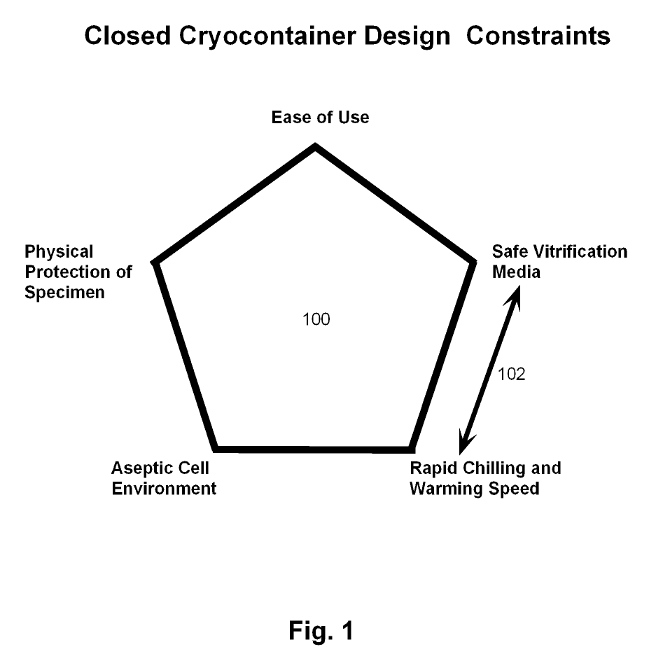

FIG. 2 is a diagram that shows the relationship between the crystallographic state of a shape memory material (exhibiting one-way shape memory) and temperature.

FIG. 3 is a diagram that shows the relationship between the crystallographic state of a shape memory material (exhibiting two-way shape memory) and temperature.

FIG. 4 illustrates features of the shuttle, sheath and assembled cryocontainer.

FIG. 4A illustrates an alternative embodiment of an end portion of the sheath

FIG. 5 illustrates the shape-shifting feature of this invention.

FIG. 6 shows the features of a temperature control bath.

FIG. 7 illustrates the vitrification process with a cryocontainer comprised of body temperature nitinol.

FIG. 8 illustrates the vitrification process with a cryocontainer comprised of superelastic nitinol.

FIG. 9 illustrates the vitrification process with a cryocontainer comprised of two-way nitinol.

FIG. 10 illustrates the vitrification process with a cryocontainer comprised of a malleable metal.

FIG. 11 shows the features of crimping tools.

FIG. 12 illustrates an alternative embodiment in which the walls of a cryocontainer are a mixture of shape memory members and non shape memory members.

FIG. 13 illustrates an embodiment that combines a cryocontainer with a deformable wall with a shape memory actuator closing device and a shape memory actuator temperature indicating device.

### DETAILED DESCRIPTION

The following detailed description discloses various embodiments and features of the invention. These embodiments and features are meant to be exemplary and not limiting.

As used herein, except for temperature and unless specifically indicated otherwise, the term “about” means within ±20% of a given value for a parameter. For temperature, “about” means ±2° C. of a given value.

A variety of biological cells can be aseptically cryopreserved (vitrified) using the present invention. One category of cells is mammalian developmental cells such as sperm, oocytes, embryos, morulae, blastocysts, and other early embryonic cells. These cells are routinely cryopreserved during assisted reproduction procedures. Another category is stem cells that are used in regenerative therapies. The broadest category is any cell that can be vitrified using a vitrification media that aligns with the available chilling speed of this invention.

### Shape Memory Effect

The shape memory effect exists in alloys of certain metals such as Ag—Cd, Au—Cd, Cu—Al—Ni, Cu—Zn—Al, Cu—Zn—Si, Cu—Zn—Sn, Cu—Sn, Cu—Zn, Fe—Pt, Fe—Mn—Si, In—Ti, Mn—Cu, Mn—Si, Ni—Ti, Ni—Al, and others. Of this group, alloys of Ni—Ti are the most commercially prevalent variant and are referred to as nitinol. Certain polymers also exhibit the shape memory phenomenon and are referred to as shape memory plastics. This invention can be implemented by a wide variety of shape memory alloys or plastics. The specific alloy or plastic to be use can be selected by those skilled in the art. To facilitate the understanding of this invention, the properties of nitinol as the shape memory material will be used in this Description to illustrate the features of this invention.

The shape memory effect is a phenomenon in which an object can exist in two different crystallographic states. The object in the first, higher temperature state is rigid with a unique defined shape. Upon cooling, this object changes to a readily deformable state. The object can be made to lose its deformability and metamorphose back to its unique defined shape by heating the material. Materials science teaches us that shuttling between these physical states is a phenomenon caused by a temperature induced phase change of the material.

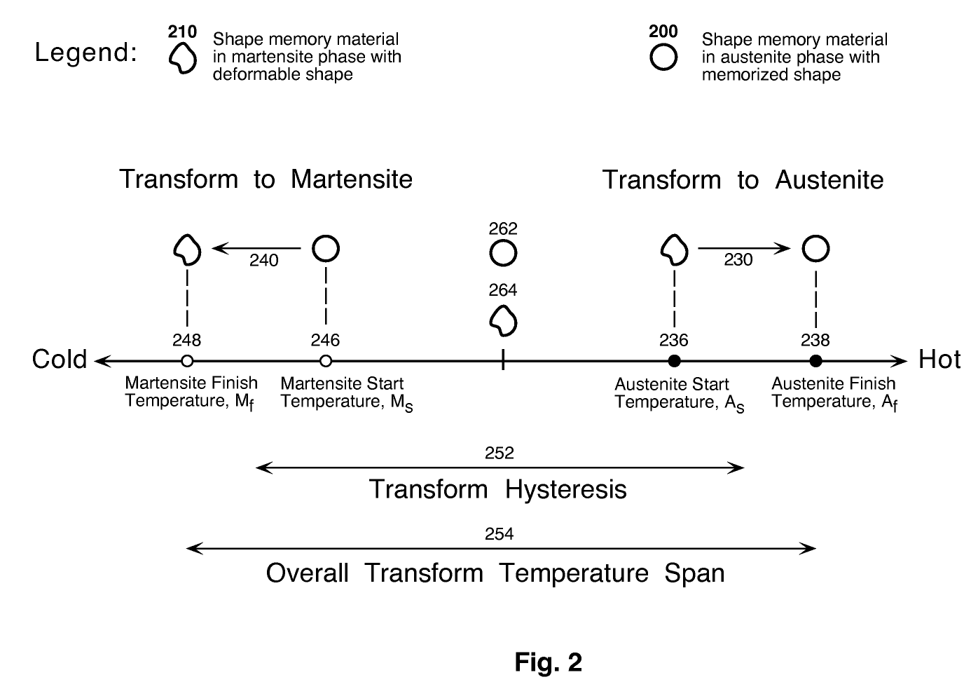

FIG. 2 is a temperature induced shape memory phase change diagram showing the behavior of “one-way” shape memory material. Shape memory materials exist in two crystallographic structures: austenite (icon 200) and martensite (icon 210). The austenite phase is characterized by stiffness and superelastic properties. The martensite phase is soft and malleable. The shape of an austenite object is referred to as the “memorized shape.” An object in the austenite phase can be transformed into martensite by cooling. As soft martensite, the object can then be deformed. This martensite object can be transformed back into austenite by heating. Upon this phase conversion, the object's shape will return (with some force) to the “memorized shape.” The transformation from a defined austenite shape to an undefined martensite shape is called one-way shape memory.

Mechanical stress can also induce a transformation from the austenite phase to the martensite phase. Once the stress is removed, however, the material reverts back to austenite. This attribute is called superelasticity, which is the ability to undergo large elastic deformations.

The word “transform” as used herein shall refer to a phase change to or from a phase with a memorized shape. The martensite to austenite transform 230 occurs over a range of temperatures from Aₛ (austenite start) 236 to A_{f}, (austenite finish) 238. By similarity, the austenite to martensite transform 240, occurs over a range of temperatures from Mₛ (martensite start) 246 to M_{f}, (martensite finish) 248. Austenite transform and martensite transform occur in different temperature bands. This phenomenon is called transformation hysteresis 252. Transformation hysteresis is the temperature spread between an object that is 50% transformed to austenite upon heating and an object that is 50% transformed back to martensite upon cooling. The overall transform temperature span 254 is the temperature range one needs to transform an object between 100% martensite and 100% austenite. For nitinol, the overall transform temperature span is approximately 50° C. An important characteristic of shape memory materials is that an object can either be in its austenite phase 262 or martensite phase 264 at a temperature between the transform temperature bands depending upon its history of heating and cooling. Methods to employ this invention utilize shape memory materials in either phase at room temperature. The desired phase can always be achieved by either: 1) warming in a warm water bath (e.g. body temperature) to transform martensite to austenite followed by cooling to room temperature or 2) chilling with a cryogenic material (e.g. dry ice, LN2, cold gaseous helium) which would be readily available for the vitrification process to transform austenite to martensite followed by warming to room temperature.

With nitinol, the transformation temperatures 246, 248, 236, and 238 are determined by the Ni to Ti atomic ratio, and the metallurgical processing of the nitinol after alloy formation. Nitinol's austenite memorized shape is configured by metallurgical processing when the material is in its austenite phase.

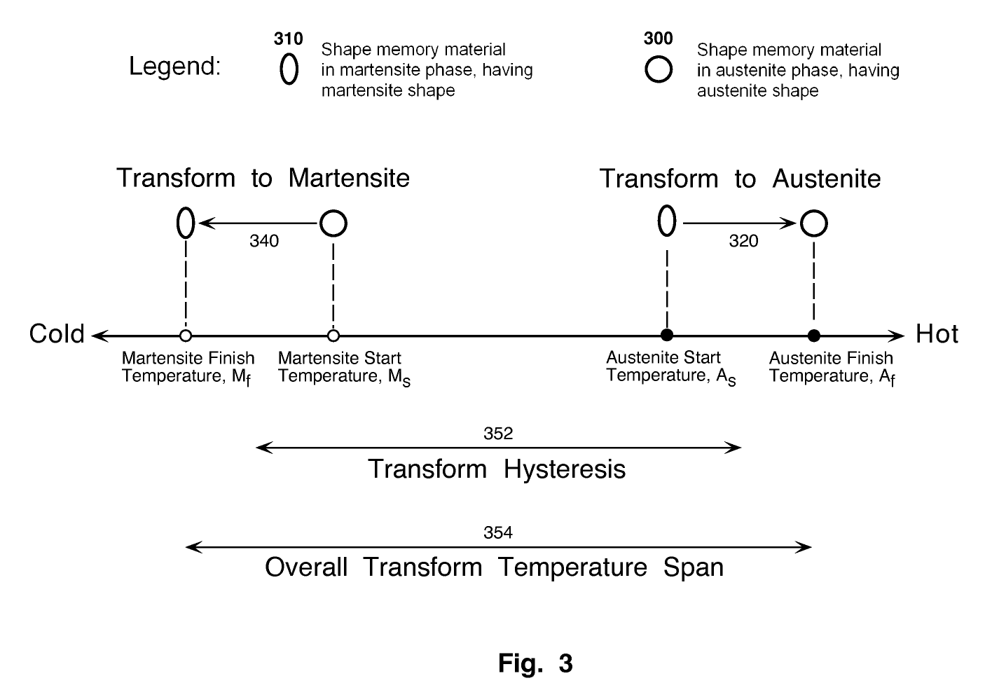

FIG. 3 is a temperature-induced shape memory phase change diagram for shape memory materials that exhibit two-way shape memory. Most shape memory materials that exhibit one-way shape memory can be trained to exhibit two-way shape memory. These materials exist in two crystallographic structures: austenite (icon 300) and martensite (icon 310). Objects fabricated from two-way shape memory materials will have two unique shapes depending on the phase. An austenite object is referred to as having the “austenite shape.” The shape of a martensite object is referred to as the “martensite shape.” Both shapes are firm and distinct. There are two “memorized shapes” in two-way shape memory versus one for one-way shape memory. The temperature transforms 320 and 340 toggles the shape memory material between the phases, and result in shape changes. Transform hysteresis 352 and overall transform temperature span 354 have a similar meaning for one-way shape memory materials.

### Principal Invention Components

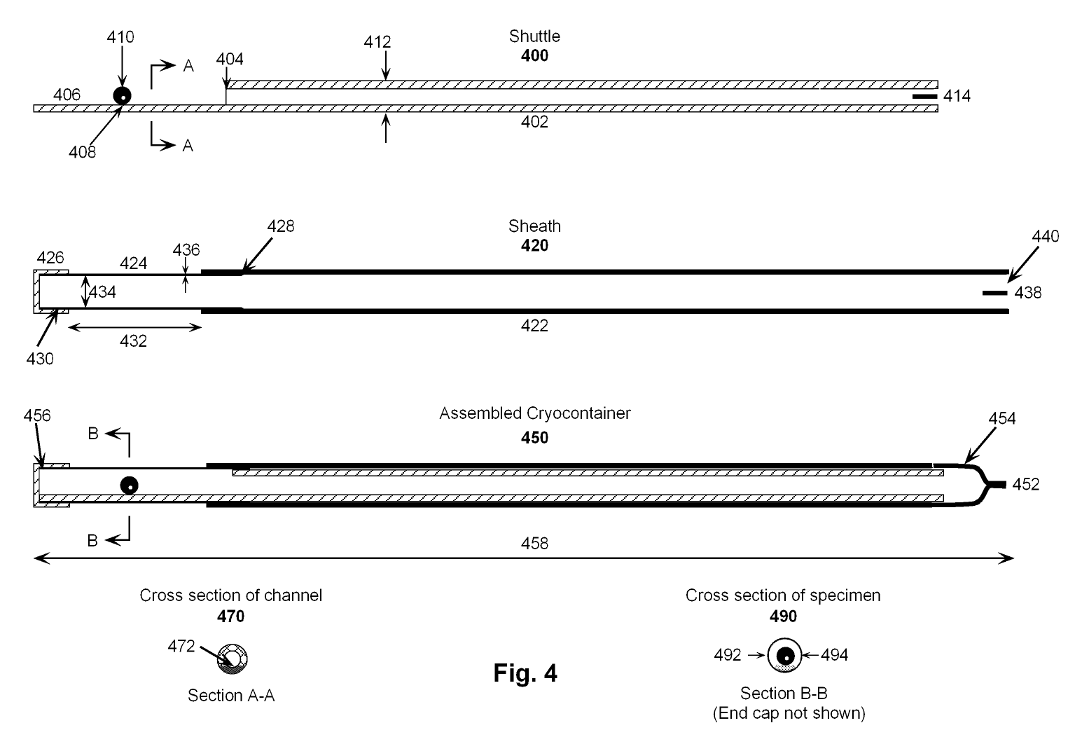

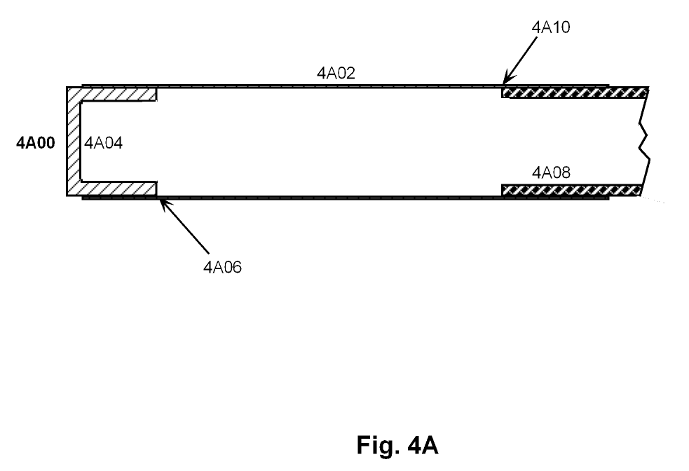

FIG. 4 illustrates longitudinal sections of generally tubular elements of an exemplary cryocontainer. The cryocontainer comprises a shuttle 400 and sheath 420. The shuttle comprises a tube 402 with a notch 404 (see also cross section 470) cut in the end to provide a channel 406 and 472. Channel indicium 408 locates where the biological specimen 410 should be placed on the channel. Channel indicium can be a groove or a printed line. The diameter 412 of the shuttle should be larger than the diameter of the biological specimen. Typical biological specimens have volume of 0.5 micro liters with a corresponding diameter of about 1 mm. A suitable diameter for the shuttle, therefore, is about 2 mm. The shuttle may comprise alignment indicia 414 to help align the shuttle with corresponding alignment indicia 438 on the sheath when the shuttle is placed within the sheath. Indicia 414 and 438 can be printed lines.

The sheath 420 comprises a tubular body 422 made of non-shape memory material, a deformable section 424 made of shape memory material and an end cap 426. The composite structure helps reduce the cost of the system since shape memory materials are relatively expensive. The tubular body is attached to the deformable section at 428. The tubular body may be disposed outboard of the deformable section with a snug fit therebetween. The joint can be strengthened by glue, welding or other joining means such that the joint forms an aseptic seal and can withstand immersion in a cryogenic fluid. The end cap is attached to the deformable section at 430. As discussed in more detail below, the shape memory material can be any material with transformation temperatures in suitable ranges. “Body temperature nitinol” is suitable.

FIG. 4A shows a longitudinal top view of the relevant heat transfer zone of an alternate embodiment 4A00 of the sheath. The deformable section 4A02 is concentrically positioned outside of the end cap 4A04 and the tubular body 4A08 with a snug fit therebetween. The joints (4A06 and 4A10) may be augmented with glue, welding or other means. The choice of a deformable section outside or inside of the tube will be determined at least in part by the relative coefficient of thermal expansions of the deformable section versus the tubular body and end cap.

The principal components of this invention may contact the biological specimen. Human reproductive cells are negatively sensitive to certain materials. Materials that do not cause such a reaction are called “non-embryotoxic.” Thus, suitable materials for the shuttle, sheath, and end cap include non-embryotoxic materials suitable for cryogenic service. Ionomer resins such a Surlyn 8921 are suitable. Our tests have shown that nitinol is non-embryotoxic and therefore is suitable as well. Nitinol can be used at cryogenic temperatures.

The length 432, diameter 434 and wall thickness 436 of the deformable section are chosen such that the deformable section may perform a shape shift cycle. A shape shift cycle is comprised of two actions: 1) the wall of the deformable section is deformed such that it touches the biological specimen and 2) the deformed wall is substantially restored to its pre-deformed shape and detaches from the biological specimen by warming to cause an austenite transform. By readily deformable, it is meant that the wall may be crimped using hand tools. Suitable deformable section diameters are 2.1 mm or greater for a shuttle diameter of 2 mm. Suitable wall thicknesses are in the range of 0.025 to 0.4 mm (preferred is 0.065 mm). Suitable lengths are in the range of 10 to 20 mm.

Assembly of the cryocontainer 450 starts with the notched end of the shuttle containing the biological specimen being advanced into opening 440 of the sheath until the end of the shuttle contacts the inner surface 456 of the end cap. Alignment indicia 414 on the shuttle and 438 on the sheath are aligned during this process. In doing so, biological specimen position indicia 492 and 494 on the deformable section (see cross section 490) are oriented with the biological specimen. Biological specimen position indicia locate suitable positions on the deformable section to crimp for the purpose of subsequent rapid heat transfer to or from the biological specimen. The open end of the sheath is then heat fused 454. This forms an aseptic seal 452 and the cryocontainer is now ready for crimping and vitrification. A sufficiently long cryocontainer prevents any heat generated by the fusing process from affecting the biological specimen. A length 458 of 4-6 cm is sufficient.

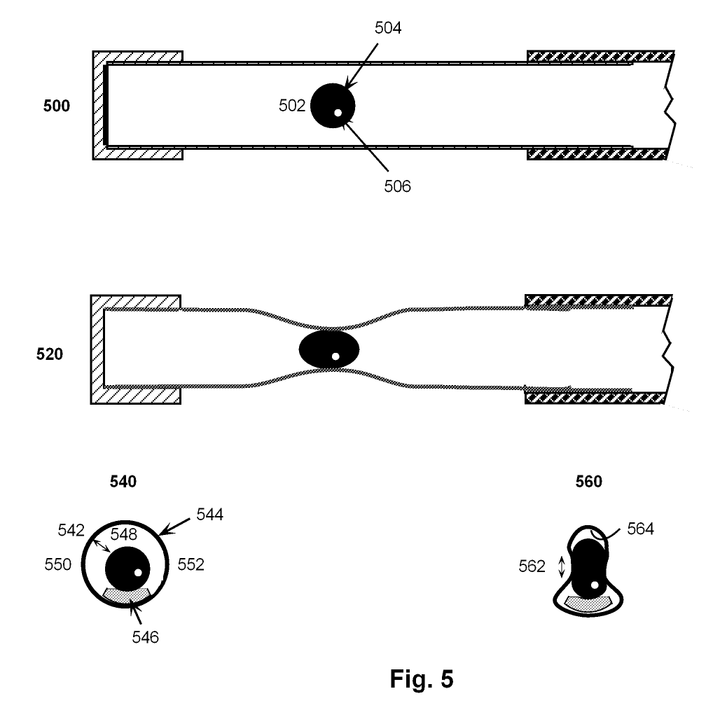

FIG. 5 shows a longitudinal top view of the relevant heat transfer zones of an assembled cryocontainer before crimping 500 and after crimping 520. Items 540 and 560 are cross sections of these items. For clarity, the channel of the shuttle is omitted from items 500 and 520. The walls of the deformable section are nitinol in its martensitic and hence malleable phase.

The drop shaped biological specimen 502 shown in item 500 comprises vitrification media 504 and one or more cells 506 that are to be cryopreserved. Referring to cross section 540, there is sufficient clearance 542 between the biological specimen 548 and the walls of the cryocontainer 544 so that the biological specimen may be easily loaded within the tubular sheath after it is placed on the channel 546 without contacting the walls of the sheath.

Referring to item 520 and corresponding cross section 560, the walls of the deformable section are crimped. Biological specimen position indicia 550 and 552 guide the crimp as they indicate points on the deformable section that are positioned over the biological specimen that also avoid the channel. The position indicia can be printed marks in the shape of cross-hairs on the deformable section. They may also be created by laser marking. Crimping can be achieved by normal means, such as the use of tweezers.

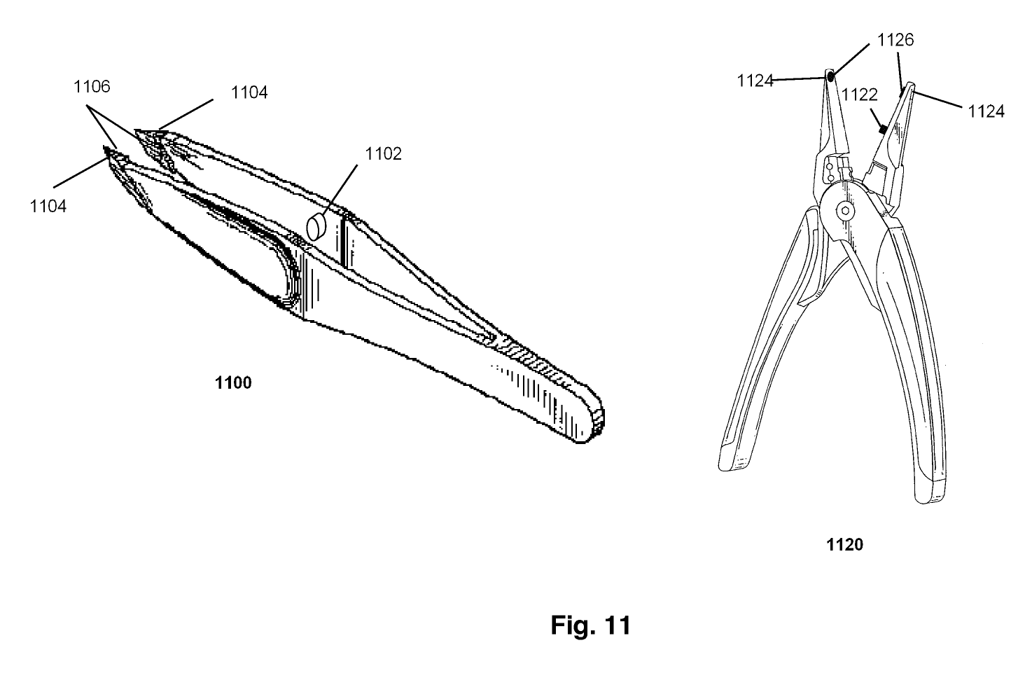

Sufficient crimping is applied such that a portion of the biological specimen 562 comes in direct contact with the sheath. In some embodiments, the modified tweezers 1100 (FIG. 11) discussed below, may be used to apply a metered amount of deformation to the deformable section to prevent overcrimping the sheath and potentially damaging the deformable section. This direct contact will allow for very high heat transfer rates between the biological specimen and the surrounding environment.

The inside wall 564 of the deformable section may be hydrophobic so that when the deformable section is returned to its original tubular shape upon warming, the biological specimen will detach therefrom and remain attached to the shuttle. If needed, the inner wall of the deformable section may be made more hydrophobic by coating it with a layer of very hydrophobic material such as polytetrafluoroethylene (marketed under the trade name Teflon) or a polyxylene polymer (marketed under the trade name Parylene).

The cryocontainer is then vitrified by exposing it a suitable cryogen such as LN2 (−196° C.). Cooling is extremely rapid (e.g. approximately one second), and the biological specimen is vitrified.

The cryocontainer may then remain in cryogenic storage for a desired period of time.

When it is necessary to recover the biological specimen, the cryocontainer is transferred from cryogenic storage to a warm water bath (e.g. 37° C., body temperature). The water bath is warm enough to transform the shape memory sheath from its deformable, martensite phase to its rigid austenite phase, i.e. the austenite transform. This causes the sheath to return to its “memorized” cylindrical shape (Item 500 and corresponding cross section 540), which is the shape optimal for unloading. The biological specimen reverts to a drop shape and clearance is restored for easy removal of the shuttle. The cryocontainer is opened, and the shuttle is removed to recover the biological specimen.

In addition to the shape-shifting feature of this invention, there are additional benefits from using nitinol as the heat transfer surface. Nitinol is stronger than the typical plastics used to fabricate cryocontainers. Additionally, its thermal conductivity is significantly higher than plastics. These attributes work together to yield a rugged deformable section that conducts heat better than plastics.

### Methods to Utilize Invention

This invention can be applied in a variety of methods. Unless noted as the exception, the examples described below utilize one-way nitinol.

Dehydration of the biological specimen with vitrification media is typically performed at room temperature, nominally 20° C. This is also the loading temperature. Rapid chilling is typically achieved with LN2 at −196° C. Warming is typically performed at 37° C. The over 200° C. temperature difference between storage and use greatly exceeds the overall transform temperature span of nitinol which is typically about 50° C. This means that any grade of nitinol, with an austenite finish temperature of approximately 37° C., can always be transformed into martensite by LN2. This holds true even if liquid propane, with an atmospheric boiling point of −42° C., is used as the cryogen.

The nitinol used to fabricate the cryocontainer needs to be deformable at room temperature and substantially restored to its memorized shape by 37° C. One method to achieve these requirements is to manufacture the nitinol with its austenite start temperature at slightly higher than room temperature and its austenite finish temperature at about body temperature, 37° C. The austenite start temperature, for example, can be in the range of 20° C. to 25° C. The austenite finish temperature can be in the range of 37° C. to 40° C. Methods to achieve this combination of austenite start temperature and austenite finish temperature include modifying the process for producing “body temperature” nitinol (e.g. Aₛ between 15° C. and 18° C., and A_{f} between 30 and 35° C.) by adjusting one or more of the ratio of nickel to titanium, the thermal processing of the alloy, or the amount of addition of a third alloying element such as copper.

Surprisingly, nitinol alloys with austenite start temperatures 2 to 4° C. below room temperature are also suitable. The alloy still retains sufficient malleability for hand crimping even though it is 2-4° C. above its austenite start temperature. Similarly, alloys with austenite finish temperature 2-4° C. above body temperature are also suitable. Nitinol will substantially recover its memorized shape for unloading upon warming to body temperature even though it is not quite up to its austenite finish temperature.

Nitinol with austenite start temperatures more than 2-4° C. below room temperature can be used if it is kept appropriately chilled and preferably crimped with a similarly chilled hand tool. Thus, standard body temperature nitinol can be used. Body temperature nitinol has an austenite start temperature of about 15° C. and an austenite finish temperature of about 33° C. Body temperature nitinol may not have sufficient malleability at room temperature, (e.g. 20° C.) for crimping with a hand tool. However, body temperature nitinol can be held artificially below room temperature so that it retains malleability during loading. A suitable holding temperature is in the range of 0-10° C. A cryocontainer fabricated from body temperature nitinol should be initially cooled below its martensite finish temperature. This can be done by placing it in a conventional freezer or plunging it into LN2. For body temperature nitinol to retain its malleability, it should be kept below its austenitic start temperature, about 15° C., up until the time it is loaded and crimped. This can be done by a number of means, such as keeping the room cold, keeping the sheath in a refrigerator, by keeping the sheath in a temperature controlled bath or combinations thereof.

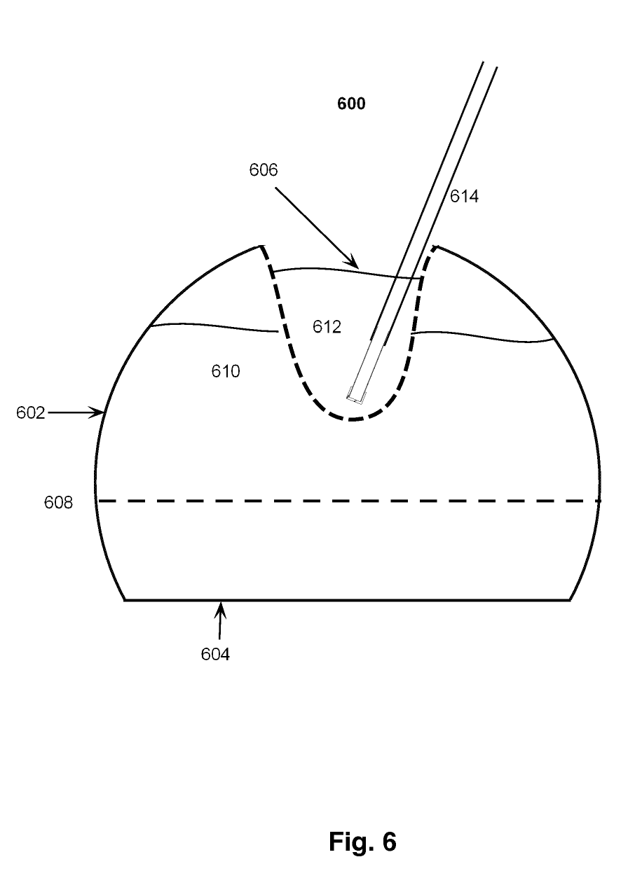

An exemplary design of a temperature controlled bath is illustrated in FIG. 6. The bath 600 comprises a roughly spherical container 602 with a flat bottom 604 and well 606 in the top. The bath may be made in two parts joined at a seam 608. A clear plastic, such as polycarbonate is a suitable material of construction. The interior of the bath is filled with a material that melts at a temperature below the austenitic start temperature. Paraffin 610 with a melting point of 10° C. is suitable.

In operation, the bath is first chilled in a refrigerator that freezes the paraffin. Prior to use, it is removed and water 612 is placed in the well. The water warms the paraffin until it begins to melt. Both the water and paraffin then thermally equilibrate at the melting point of the paraffin. A sheath 614 that has been chilled below its martensite finish temperature can then be immersed in the well and will be maintained at the bath temperature. If crimping is done with a tool such as tweezers, the tool can be similarly equilibrated at the water bath temperature so that it does not bring the deformable section above the austenitic start temperature when it touches it.

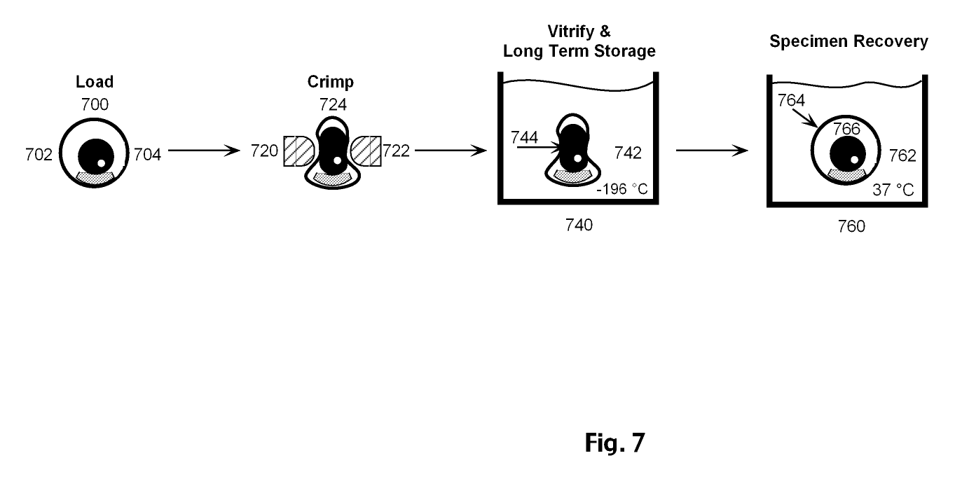

Referring to FIG. 7, when it is time to vitrify a biological specimen, the sheath is removed from the well, and the shuttle with the biological specimen is placed into the sheath. The relevant vitrification heat transfer zone is shown as cross section 700. The biological specimen is hidden from view so biological specimen position indicia 702 and 704 on the deformable section indicate the optimal points to form a crimp. A handheld tool, such as a modified tweezers with crimping points 720 and 722, crimps the cryocontainer to form cross section 724. Cross section 724 shows the cryocontainer just after the crimp. The malleable body temperature nitinol retains its crimped configuration without external forces. Crimping is a fast process such that the body temperature nitinol does not warm to a temperature that compromises its malleability. The entrance of the cryocontainer is then sealed by conventional means, such as heat sealing.

The cryocontainer is then placed in a cryogenic bath 740 that contains, for example, liquid nitrogen 742 at −196° C. Cooling is extremely rapid (e.g. about one second), and the biological specimen 744 is vitrified.

The cryocontainer may then remain in cryogenic storage for a desired period of time.

When it is necessary to recover the vitrified biological specimen, the cryocontainer is transferred from the liquid nitrogen bath to a warm water bath 760 containing water 762 at 37° C., body temperature. The water bath is warm enough to transform the shape memory sheath from its deformable, martensite phase to its rigid austenite phase. This causes the sheath to return to its “memorized” cylindrical shape 764. Thus the biological specimen 766 is safely warmed and clearance is again provided for easy removal of the shuttle.

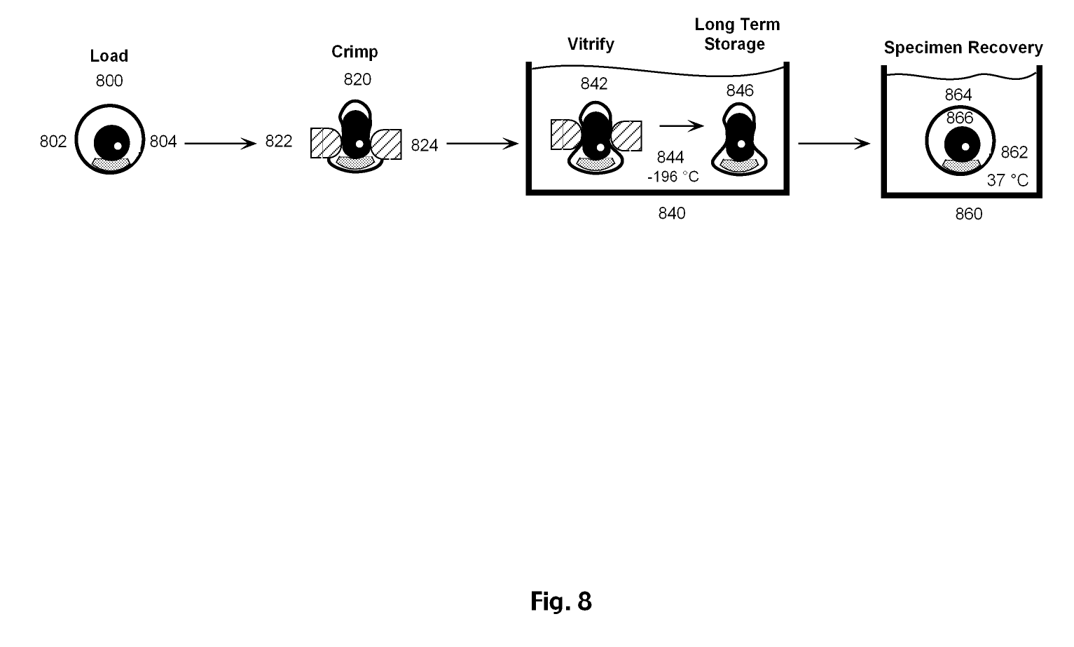

Referring to FIG. 8, nitinol alloys with low austenitic finish temperatures, such as about 10° C., can be used by taking advantage of nitinol's superelastic properties when in the austenite phase. Cross section 800 shows a cryocontainer fabricated from nitinol that is austenite at room temperature. A shuttle with biological specimen is assembled with the sheath as described above. An aseptic seal is then formed. Biological specimen position indicia 802 and 804 indicate optimal crimping points on the cryocontainer. As compared to martensite, austenitic nitinol requires higher forces to achieve deformation. Thus, rather than tweezers, a pair of modified pliers are needed to deform the austenite. The contact points 822 and 824 of such a pair of pliers then crimps the cryocontainer to form cross section 820. The pliers must continuously maintain their crimping force for the cryocontainer to remain crimped.

The clenched pair of pliers that holds the crimped cryocontainer 842 is then placed in a cryogenic bath 840 that contains LN2 844. Cooling is conducted from the LN2 through the pliers' contact points to achieve vitrification. Simultaneously, the deformable section is transformed into martensite. The pliers can then be removed. The cryocontainer 846 retains its crimped shape, a shape optimal for heat transfer, which will come into play during warming. The cryocontainer is now ready for long term cryogenic storage.

To recover the vitrified biological specimen, the cryocontainer is transferred from the liquid nitrogen bath to a warm water bath 860 (e.g. 37° C., body temperature). The water 862 is warm enough to transform the shape memory sheath from its deformable, martensite phase to its rigid austenite phase. This causes the sheath to return to its “memorized” cylindrical shape 864. Thus the biological specimen 866 is safely warmed and clearance is again provided for easy removal of the shuttle.

### Two-Way Shape Memory Alloy

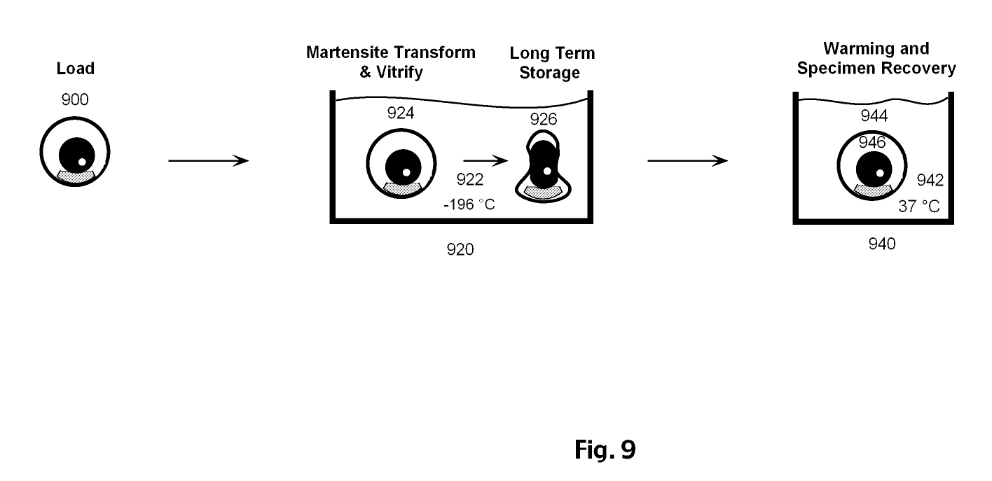

Referring to FIG. 9, this invention can also be implemented with a cryocontainer fabricated from two-way nitinol. An example is a material having a martensite finish temperature of −10° C. and an austenite finish temperature of 37° C. A sheath made from this material can have a cylindrical austenite shape and a crimped martensite shape. The shuttle with biological specimen is assembled with the sheath as described above. The aseptic seal is formed. Cross section 900 shows a cryocontainer fabricated from two-way nitinol ready for vitrification. No crimping tools are needed so biological specimen position indicia are not needed.

To vitrify, the cryocontainer is placed in a cryogenic bath 920 that contains LN2 922. Cooling induces a martensite transform that shape-shifts the deformable section from its austenite shape 924 to its martensite shape 926. This shape is optimal for heat transfer and will come into play during warming. Simultaneously with the phase change, the rapid cooling vitrifies the biological specimen. The cryocontainer can then be placed into long term cryogenic storage.

Recovery of the vitrified biological specimen follows the same steps as the other embodiments. The cryocontainer is transferred from the liquid nitrogen bath to a warm water bath 940 (e.g. 37° C., body temperature). The water 942 is warm enough to invoke the austenite transform which shape-shifts the deformable section back to its memorized austenite shape 944 (cylinder). Thus the biological specimen 946 is safely warmed and clearance is again provided for easy removal of the shuttle.

### Malleable Metals

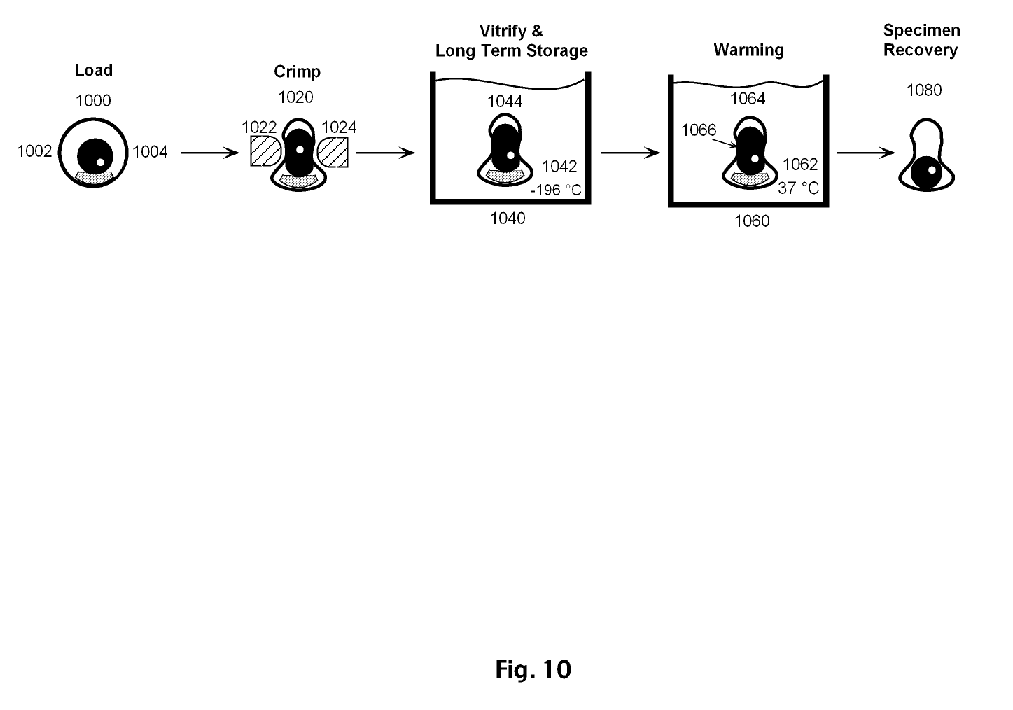

Referring to FIG. 10, malleable metals such as gold, silver, copper, tin, and aluminum can be readily deformed with hand tools such as tweezers. They also have high thermal conductivity that contributes to rapid heat transfer. A sheath can be fabricated with a malleable metal deformable section. A shuttle with biological specimen is assembled with this sheath. The aseptic seal is formed. Cross section 1000 shows a cryocontainer in which the deformable section is fabricated from a malleable metal. Biological specimen position indicia 1002 and 1004 indicate optimal crimping points to achieve a shape optimal for heat transfer. A hand tool, such as a modified tweezers with crimping points 1022 and 1024, crimps the cryocontainer to form cross section 1020. The malleable metal retains its crimped configuration without external forces. The crimped cryocontainer is then placed in a cryogenic bath 1040 that contains LN2 1042. Cooling is extremely rapid (e.g. about one second), and the biological specimen 1044 is vitrified. The cryocontainer is now ready for long-term cryogenic storage.

To recover the vitrified biological specimen, the cryocontainer 1064 is transferred from the LN2 to a warm water bath 1060 (e.g. 37° C., body temperature). The water 1062 safely warms the biological specimen 1066.

The malleable metal deformable section does not by temperature change revert back to its cylindrical shape. One way to restore the shape is to apply an air pressure source at the entrance 440 (FIG. 4) of the sheath. Pressure is applied to the inside cavity of the sheath which mechanically restores the crimped malleable metal tube to its original cylindrical shape. Pressures of 1-50 psig are suitable with preferred pressures of 1-15 psig. Human cells have been shown to withstand short exposures to high (several hundreds of bar) pressure. Therefore, the design parameters of the sheath to allow outward deformation of the crimped sheath with internal air pressure will not normally be limited by the biological specimen. Clearance is restored allowing easy removal of the shuttle for recovery of the biological specimen.

An alternate recovery method is to remove the shuttle from the warmed cryocontainer 1080, leaving behind the biological specimen. A fine needle syringe can then be inserted into the entrance of the sheath to irrigate the internal cavity with a flushing fluid. The biological specimen can be found in the drained flushing fluid if it is not on the shuttle.

### Crimping Hardware

FIG. 11 illustrates modified tweezers 1100 and modified pliers 1120 which may be used to crimp a deformable section. The tweezers (pliers) comprise a stop 1102 (1122) to insure that the crimp is to a predetermined depth. They also comprise crimping indicia 1104 (1124) to help the user properly align them with the biological specimen position indicia on a deformable section when crimping. Jaws 1106 (1126) may be modified to impose a predetermined shape upon the deformable section during crimping. The higher mechanical advantage of the pliers will exert more crimping force than tweezers. This additional force is useful in crimping superelastic nitinol alloys.

### Alternate Sheath Configurations and Composite Materials

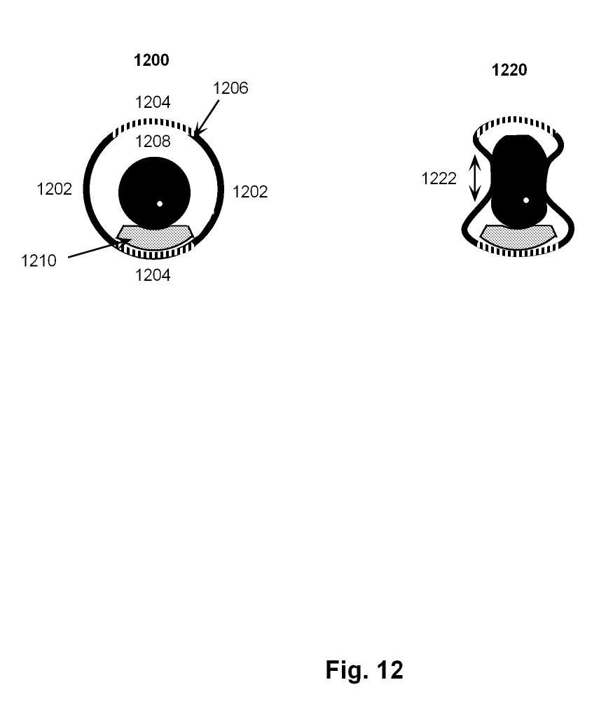

FIG. 12 shows a cross section of a cryocontainer 1200 with composite walls fabricated with two opposing nitinol parts 1202 and two non-nitinol parts 1204. Reducing the amount of nitinol helps keep the materials cost low. The non-nitinol sides are fabricated from non-embryotoxic materials that are suitable for cryogenic temperatures. The four sides of the cryocontainer are joined at four places represented by 1206. Within the cryocontainer is a biological specimen 1208 placed on a channel 1210. The composite wall cryocontainer has a memorized shape suitable for loading and unloading. Similar to the other embodiments described herein, the nitinol in this composite cryocontainer can be either malleable martensite or austenite. Therefore, by using crimping tools, it can be crimped to form cross section 1220. This shape contacts 1222 the biological specimen for improved heat transfer. In warming to 37° C., this cryocontainer will revert to its cylindrical memorized shape. The memorized shape restores clearances that allow for easy biological specimen recovery.

### General Considerations

Shape memory devices fabricated from nitinol are suitable for 8% recoverable strain. Copper based shape memory materials are suitable for 12% recoverable strains. The invention is functional using materials that can withstand either recoverable or non-recoverable strains of at least 1% without rupturing. Higher recoverable strains lead to deeper crimping for better heat transfer while still returning to their memorized shapes. This invention can be applied to non-circular shapes that can more efficiently utilize the available recoverable strain to achieve higher chilling speeds.

### Shape Memory Polymers

Shape memory polymers are polymers that exhibit a shape memory phenomenon. However, the phenomenon in polymers does not arise from two crystallographic states as in shape memory alloys with two transform temperature bands. Only one characteristic temperature, called the glass transition temperature, T_{g}, is needed to understand shape memory polymers. The memorized shape is established during fabrication at a temperature above T_{g}. The polymeric part can then be deformed to a different shape and cooled below T_{g}. The part will retain its deformed shape as long as it remains below T_{g}. When the part is heated above, T_{g}, it reverts to its memorized shape. Thus, a cryocontainer can be fabricated with a shape memory polymer deformable section and will function as a shape-shift vitrification cryocontainer by using the methods taught in FIG. 8. In some embodiments suitable shape memory polymers may be plastic. Veriflex® is a suitable shape memory polymer.

A cryocontainer fabricated from shape memory polymers having a T_{g} of 15° C. can be crimped at room temperature and then held and vitrified. It will retain its crimped shape at cryogenic temperatures. When the cryocontainer is heated above T_{g}, it will return to its uncrimped shape and the biological specimen can be removed.

### Cryocontainer With Deformable Section, Shape Memory Actuator Closure Device and Shape Memory Actuator Temperature Indicator

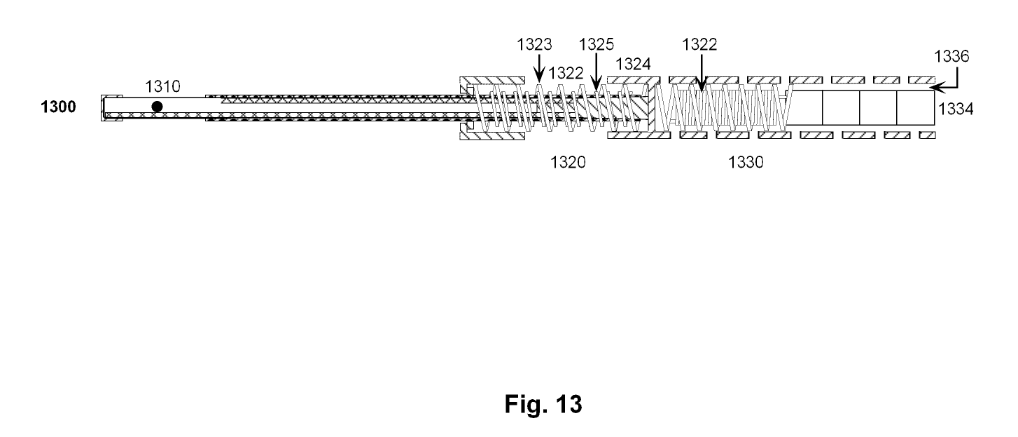

FIG. 13 illustrates a cryocontainer 1300 with a deformable section 1310, shape memory actuator closing device 1320 and a shape memory actuator temperature indicator 1330.

The shape memory actuator closure device comprises a shape memory actuator 1322 and an end cap 1324. The shape memory actuator comprises a shape memory spring 1323 and a conventional material bias spring 1325. The shape memory spring has a relatively expanded memorized shape and is stronger than the bias spring at room temperature. The end cap, therefore, is pushed away from the end of the cryocontainer at room temperature and can be removed allowing for loading and unloading of the shuttle and biological specimen. When the actuator is chilled, however, such as by grasping with chilled tongs, the shape memory spring transforms to martensite, becomes weaker than the bias spring and the bias spring pulls the end cap snugly against the end of the cryocontainer. Thus a continual closing force is applied to the cryocontainer helping to seal it during vitrification and storage.

The temperature indicating device comprises a shape memory actuator 1322, alert rod 1334 and outer chamber 1336. The shape memory actuator is normally collapsed at cryogenic temperatures and expanded at higher temperatures, such as the devitrification temperature of −130 C. Thus if the cryocontainer is removed from cryogenic storage for inspection or other reason, the user will get a warning of potential devitrification due to the warming of the actuator and movement of the temperature alert rod out from the chamber.

### Conclusion

While the disclosure has been described with reference to one or more different exemplary embodiments, it will be understood by those skilled in the art that various changes may be made and equivalents may be substituted for elements thereof without departing from the scope of the disclosure. In addition, many modifications may be made to adapt to a particular situation without departing from the essential scope or teachings thereof. Therefore, it is intended that the disclosure not be limited to the particular embodiment disclosed as the best mode contemplated for carrying out this invention.

1. A cryocontainer for vitrifying a biological specimen, said cryocontainer comprising:
a. a shuttle, said shuttle comprising a channel for holding said biological specimen; and
b. a sheath, said sheath comprising a deformable section,
wherein said shuttle and said sheath are dimensioned such that at least a portion of said channel is located within said deformable section when said shuttle is loaded into said sheath, and wherein said deformable section comprises a shape memory material.

2. The cryocontainer of claim 1 wherein said shape memory material is a plastic.

3. The cryocontainer of claim 1 wherein said shape memory material is a metal.

4. The cryocontainer of claim 3 wherein said metal is a nitinol alloy and said nitinol alloy has:
a. an austenite start temperature in the range of 10° C. to 25° C.; and
b. an austenite finish temperature in the range of 30° C. to 45° C.

5. The cryocontainer of claim 4 wherein:
a. said austenite start temperature is in the range of 20° C. to 25° C.; and
b. said austenite finish temperature is in the range of 35° C. to 40° C.

6. The cryocontainer of claim 3 wherein the austenite finish temperature of said metal is less than 25° C.

7. The cryocontainer of claim 1 wherein:
a. said shuttle has a diameter of about 2 mm;
b. said deformable section has a tubular shape;
c. said deformable section has an internal diameter at least 0.1 mm greater than said shuttle diameter;
d. said deformable section is longer than 10 mm; and
e. the wall of said deformable section has a thickness in the range of 0.025 to 0.4 mm.

8. The cryocontainer of claim 7 wherein said wall of said deformable section may be crimped without rupturing by a hand tool to the point where the minimum internal spacing of said deformable section is about 1 mm.

9. The cryocontainer of claim 1 wherein:
a. biological specimen position indicia are presented on the outside surface of said deformable section to indicate where the deformable section should be crimped; and
b. alignment indicia are presented on the shuttle and the sheath to indicate how the two should be aligned when assembled.

10. The cryocontainer of claim 1 wherein:
a. the surface of said channel is non-embryotoxic and;
b. the internal surface of said deformable section is non-embryotoxic and hydrophobic.

11. The cryocontainer of claim 1 which further comprises:
a. a shape memory actuator closure device; and
b. a shape memory actuator temperature indicator.

12. A cryocontainer for vitrifying a biological specimen, said cryocontainer comprising:
a. a shuttle, said shuttle comprising a channel for holding said biological specimen; and
b. a sheath, said sheath comprising a deformable section,
wherein said shuttle and said sheath are dimensioned such that at least a portion of said channel is located within said deformable section when said shuttle is loaded into said sheath, and wherein said deformable section comprises a malleable metal material.

13. The cryocontainer of claim 12 wherein the malleable metal can be gold, silver, copper, tin, or aluminum.

14. The cryocontainer of claim 12 wherein:
a. said shuttle has a diameter of about 2 mm;
b. said deformable section has a tubular shape;
c. said deformable section has an internal diameter at least 0.1 mm greater than said shuttle diameter;
d. said deformable section is longer than 10 mm; and
e. the wall of said deformable section has a thickness in the range of 0.025 to 0.4 mm.

15. The cryocontainer of claim 14 wherein said wall of said deformable section may be crimped without rupturing by a hand tool to the point where the minimum internal spacing of said deformable section is about 1 mm.

16. The cryocontainer of claim 12 wherein:
a. biological specimen position indicia are presented on the outside surface of said deformable section to indicate where the deformable section should be crimped; and
b. alignment indicia are presented on the shuttle and the sheath to indicate how the two should be aligned when assembled.

17. The cryocontainer of claim 12 wherein:
a. the surface of said channel is non-embryotoxic and;
b. the internal surface of said deformable section is non-embryotoxic and hydrophobic.

18. The cryocontainer of claim 12 which further comprises:
a. a shape memory actuator closure device; and
b. a shape memory actuator temperature indicator.

19. A method of vitrifying a biological specimen, said method comprising the steps of:
a. placing said biological specimen inside a cryogenic container, said cryogenic container comprising a deformable wall;
b. crimping said deformable wall such that it contacts said biological specimen and increases the heat transfer rate thereto; and
c. contacting said deformable wall with a cryogenic substance such that said biological specimen is vitrified.

20. The method of claim 19 wherein said deformable wall comprises nitinol with an austenite start temperature in the range of 20° C. to 25° C. and wherein said step of crimping said deformable wall is performed at about 20° C.

21. The method of claim 19 wherein said deformable wall comprises body temperature nitinol and wherein said method further comprises the steps of:
a. chilling said deformable wall to a temperature below the martensitic finish temperature of said nitinol prior to said insertion of said biological specimen;
b. chilling a crimping tool to a temperature below the austenitic start temperature of said nitinol; and
c. performing said step of crimping said deformable wall using said chilled crimping tool.

22. The method of claim 21 wherein said crimping tool is a hand tool, said deformable wall comprises indicia and said crimping tool is aligned with said indicia during said step of crimping.

23. The method of claim 19 wherein said deformable wall comprises a two-way shape memory metal wherein the austenitic shape of said two-way shape memory metal is an open shape and the martensitic shape of said two-way shape memory metal is a crimped shape and wherein said step of said crimping comprises cooling said deformable wall below the martensitic start temperature of said two-way shape memory metal.

24. The method of claim 19 which further comprises the steps of:
a. warming said biological specimen to 37° C.; and
b. expanding said deformable section using gas pressure such that said deformable wall detaches from said biological specimen.

25. The method of claim 19 wherein said deformable wall is nitinol in its austenitic phase at room temperature and which further comprises the steps of:
a. crimping and holding said deformable wall with a pair of pliers; and
b. holding said deformable section in a crimped position during said step of contacting said deformable wall with said cryogenic substance.

26. The method of claim 19 wherein said deformable wall is a malleable metal and comprises indicia and said crimping is achieved with a crimping tool aligned with said indicia.

27. A biological specimen, said biological specimen having been processed by a method comprising the steps of:
a. placing said biological specimen inside a cryogenic container, said cryogenic container comprising a deformable wall;
b. crimping said deformable wall such that it contacts said biological specimen; and
c. contacting said deformable wall with a cryogenic substance such that said biological specimen is vitrified.

28. The biological specimen of claim 27 wherein said deformable wall comprises nitinol and wherein said biological specimen has been further processed by the step of warming said biological specimen by immersing said deformable wall in a water bath at approximately body temperature such that said deformable wall transforms into a memorized shape such that said biological specimen may be removed from said cryogenic container without touching said wall.

29. The biological specimen of claim 28 wherein the inside surface of said deformable wall is coated with Teflon or the like such that said biological specimen dewets from said wall when said deformable wall transforms to its memorized shape.
